(19) United States
(12) Patent Application Publication
(10) Pub. No.: US 2021/0212623 A1
(43) Pub. Date: Jul. 15, 2021

(54) METHODS, SYSTEMS, AND DEVICES FOR CONTROLLING TEMPERATURE IN A BIOELECTRIC MEASUREMENT SYSTEM

(71) Applicant: Vine Medical LLC, Saint George, UT (US)

(72) Inventor: Phillip W. Dietz, Saint George, UT (US)

(73) Assignee: Vine Medical LLC, Saint George, UT (US)

(21) Appl. No.: 17/150,964

(22) Filed: Jan. 15, 2021

Related U.S. Application Data
(60) Provisional application No. 62/961,616, filed on Jan. 15, 2020.

(51) Int. Cl. A61B 5/24; A61B 5/00

(52) U.S. Cl. CPC A61B 5/24; A61B 2562/0271; A61B 5/746

(57) ABSTRACT

Systems, methods, and devices for measuring temperature in an automated bioelectric measurement system through a thermal sensor and using temperature readings to regulate fan activity to improve accuracy of bioelectric measurements. A bioelectric measurement device may include a housing, a conductive tip disposed at a distal end of the housing, a motor within the housing that applies a motor output force to the conductive tip, a thermal sensor that takes a temperature measurement of an inside of the housing, a fan that cools the inside of the housing including the motor, and a controller that controls the fan based on the temperature measurement from the thermal sensor.

### CROSS-REFERENCE TO RELATED APPLICATIONS

This application claims the benefit of U.S. Provisional Application No. 62/961,616, filed Jan. 15, 2020, titled, “METHODS, SYSTEMS. AND DEVICES FOR CONTROLLING TEMPERATURE IN A BIOELECTRIC MEASUREMENT SYSTEM,” which is incorporated herein by reference in its entirety, including but not limited to those portions that specifically appear hereinafter, the incorporation by reference being made with the following exception: In the event that any portion of the above-referenced provisional application is inconsistent with this application, this application supersedes the above-referenced provisional application.

### TECHNICAL FIELD

The disclosure is directed to controlling the temperature in a bioelectric measurement system comprising an electrodermal probe for taking bioelectric readings and measurements of a test subject, and more particularly directed to systems, methods, and devices for measuring temperature in an automated bioelectric measurement system through a thermal sensor and using temperature readings to regulate fan activity to improve accuracy of bioelectric measurements.

### BACKGROUND

The electrical conductance of body tissue can be measured and analyzed to gather information about a body's condition and to aid in diagnosing certain conditions. One form of measuring electrical conductance of body tissue is Electroacupuncture According to Voll (EAV). EAV and other electrical conductance diagnostic systems measure conductance levels at meridian points of the body. These electrical conductance diagnostic systems are used by some health practitioners to gain additional insight into the body's make up, condition, and compatibility with certain supplements or materials, and whether certain pathogens or toxins reside in the body, whether certain dental conditions are present in the body, and more.

EAV and other electrical conductance assessment systems often utilize an electrodermal probe in contact with the body tissue of a test subject in order to measure and/or test electrical conductance of the test subject's body tissue at meridian points of the body. Current devices for measuring skin conductivity consist of a conductive metal tip on the electrodermal probe that is used to measure conductivity of meridian points and other tissues of a body of a test subject. A grip area is located adjacent to the conductive metal tip, allowing a technician to grip the device while taking measurements of the test subject. The entire device is enclosed in a non-conductive housing to prevent the measurements from being compromised by outside electrical influences. A cable connects the device to an EAV or other system to receive the measurement and calculate the skin conductivity.

Because many bio-conductance measurement devices do not contain a printed circuit board or processor, they need to be connected to an EAV device or similar device to perform skin conductivity testing. Although the device is enclosed in a non-conductive housing to limit contamination during measurements and compromising of the results, the conductive metal tip is not shielded from contact with other objects that could impact the conductivity measurement.

Further, because many devices are manually controlled by the technician performing the testing, conductivity measurements can vary from technician to technician while tests are being performed due to variability in the different forces applied by different technicians as they measure meridian points and other tissues of a test subject. The differences in application force may lead to inaccurate and inconsistent measurements between technicians and may potentially lead to misdiagnosis and/or treatment.

To minimize compromised measurements due to different forces applied by different technicians in bio-conductance testing, automated bio-conductance measurement devices and bioelectric measurement systems that automate the application of force with the conductive tip of the electrodermal probe against skin/tissue of a test subject's body using a motor with a computerized control system. Although these automated systems reduce variability and inaccuracy in measurements, the control system and/or components of the motor can become compromised by heat as the motor functions and temperature within the system increases. Rising temperatures within the system lead motor components, and thereby the motor, to not operate properly, which in turn leads to unpredictability and variability in the application of force by the conductive metal tip to the tissue/body of the test subject that is accomplished by the motor. Variability in the application of force leads to compromised, variable, and inaccurate conductance measurements/results, which are not desirable in bio-conductance measurement devices and bioelectric measurement systems.

In light of the foregoing, disclosed herein are systems, methods, and devices for better maintaining proper temperature and force, and improved operation of an automated bioelectric measurement system to improve accuracy and consistency of conductance measurements taken of a test subject.

### BRIEF DESCRIPTION OF THE DRAWINGS

Non-limiting and non-exhaustive implementations of the disclosure are described with reference to the following figures, wherein like reference numerals refer to like parts throughout the various views unless otherwise specified. Advantages of the disclosure will become better understood with regard to the following description and accompanying drawings where:

FIG. 1A is a side view of an embodiment of a manual bioelectric measurement system including a conductive metal tip, a grip area, and a cord connecting the device to an EAV or similar device;

FIG. 1B illustrates a perspective view of a manual bioelectric measurement system of FIG. 1A;

FIG. 2A illustrates a side view of an embodiment of an automated bioelectric measurement system including a conductive metal tip, an isolating hood, a non-conductive probe body, and a cable connecting the system to the EAV device;

FIG. 2B illustrates a side view of an embodiment of an automated bioelectric measurement system with a portion of a housing removed to reveal interior components of the system, including a thermal sensor, a motor, a fan, and one or more processors/controllers in communication with an electrodermal probe and an EAV device;

FIG. 3 illustrates an embodiment of a method for measuring temperature in an automated bioelectric measurement system and using an algorithm to determine whether to turn a fan on or off based on temperature measurements;

FIG. 4 illustrates an embodiment of a method for measuring temperature in an automated bioelectric measurement system and using an algorithm to determine the fan speed necessary to reduce internal temperatures within the automated bioelectric measurement system;

FIG. 5 illustrates an embodiment of a method for measuring temperature in an automated bioelectric measurement system and using an algorithm to calculate the compensation required to maintain constant motor output force based on the temperature measurement;

FIG. 6 illustrates an embodiment of a method for measuring temperature in an automated bioelectric measurement system and using an algorithm to evaluate the sufficiency of fan and motor compensations and turning off the automated bioelectric measurement system to prevent overheating if the adjustments are not sufficient to reduce internal temperatures;

FIG. 7 illustrates an embodiment of a method for measuring temperature in an automated bioelectric measurement system and using an algorithm to determine whether to turn a fan on or off based on temperature measurements; and

FIG. 8 is a block diagram illustrating an example computing device.

### DETAILED DESCRIPTION

Disclosed herein are systems, methods, and devices for measuring temperature in an automated bioelectric measurement system through a thermal sensor and using temperature readings to regulate fan activity, motor output force, and/or bioelectric measurement device operation to improve accuracy of bioelectric measurements. The thermal sensor may be used in conjunction with an electrical conductance diagnostic system such as an Electroacupuncture According to Voll (EAV) or other electrodermal sensor systems.

In the following description, for purposes of explanation and not limitation, specific techniques and embodiments are set forth, such as particular techniques and configurations, in order to provide a thorough understanding of the system and device disclosed herein. While the techniques and embodiments will primarily be described in context with the accompanying drawings, those skilled in the art will further appreciate that the techniques and embodiments may also be practiced in other similar devices.

For the purposes of promoting an understanding of the principles in accordance with the disclosure, reference will now be made to the embodiments illustrated in the drawings and specific language will be used to describe the same. It will nevertheless be understood that no limitation of the scope of the disclosure is thereby intended. Any alterations and further modifications of the inventive features illustrated herein, and any additional applications of the principles of the disclosure as illustrated herein, which would normally occur to one skilled in the relevant art and having possession of this disclosure, are to be considered within the scope of the disclosure claimed.

Before the systems, methods, and devices for measuring temperature in an automated bioelectric measurement system through a thermal sensor and using temperature readings to regulate fan activity, motor output force, and/or bioelectric measurement device operation are disclosed and described, it is to be understood that this disclosure is not limited to the particular structures, configurations, process steps, and materials disclosed herein as such structures, configurations, process steps, and materials may vary somewhat. It is also to be understood that the terminology employed herein is used for the purpose of describing particular embodiments only and is not intended to be limiting since the scope of the disclosure will be limited only by the appended claims and equivalents thereof.

In describing and claiming the subject matter of the disclosure, the following terminology will be used in accordance with the definitions set out below.

It must be noted that, as used in this specification and the appended claims, the singular forms “a,” “an,” and “the” include plural referents unless the context clearly dictates otherwise.

As used herein, the terms “comprising,” “including,” “containing,” “characterized by,” and grammatical equivalents thereof are inclusive or open-ended terms that do not exclude additional, unrecited elements or method steps.

As used herein, the phrase “consisting of” and grammatical equivalents thereof exclude any element or step not specified in the claim.

As used herein, the phrase “consisting essentially of” and grammatical equivalents thereof limit the scope of a claim to the specified materials or steps and those that do not materially affect the basic and novel characteristic or characteristics of the claimed disclosure.

Reference will now be made in detail to the exemplary embodiments, examples of which are illustrated in the accompanying drawings. Wherever possible, the same reference numbers are used throughout the drawings to refer to the same or like parts. It is further noted that elements disclosed with respect to particular embodiments are not restricted to only those embodiments in which they are described. For example, an element described in reference to one embodiment or figure, may be alternatively included in another embodiment or figure regardless of whether or not those elements are shown or described in another embodiment or figure. In other words, elements in the figures may be interchangeable between various embodiments disclosed herein.

In the following description of the disclosure, reference is made to the accompanying drawings, which form a part hereof, and in which is shown by way of illustration specific implementations in which the disclosure may be practiced. It is understood that other implementations may be utilized, and structural changes may be made without departing from the scope of the disclosure.

Electroacupuncture According to Voll (EAV) devices may be deployed to measure conductance levels at meridian points in a body. An EAV device is a sensitive ohm meter for measuring resistance in the body. The resistance of a material, tissue, meridian pathway, and so forth can be assessed to calculate the conductivity of the material, tissue, or meridian pathway. A material with a lower resistance measurement will have a higher conductivity.

To detect resistance, an ohm meter (such as an EAV device) applies a small direct current flow through a material. Resistance measures the relative difficulty for current to flow through the material. Electrical conductors allow current to flow easily and have a correspondingly low resistance. Electrical insulators restrict current flow through and have a correspondingly high resistance. Ohm's Law applies to materials with a proportional relationship between voltage, current, and resistance according to:

V=RI

where V is voltage (measured in volts), I is current (measured in amps) and R is resistance (measured in ohms). Conductivity is the reciprocal of resistivity, expressed mathematically as 1/R and indicates a degree to which a specified material conducts electricity.

Human tissue generally has a resistance of about 98,000 Ohms between the tissue and ground. Meridian points have a general resistance of about 5,000 Ohms between the meridian point and ground. This means that meridian points throughout the human body are about twenty times more conductive than the tissue surrounding the points. This large differential in conductivity makes it possible to locate meridian points and to be very consistent in verifying the points with an EAV device.

EAV testing may be carried out in a plurality of different ways. Traditional EAV testing or resistance measuring of meridian points may be done by having a subject contact a metal rod (or other electrodes, hand masses, or grounding devices) with a part of the subject's body while point measurements are taken elsewhere on the subject's the body with a probe of the EAV device. The point measurements may use an electrically conductive tip to contact the meridian point and the measurements are taken and recorded while pressure is applied to the metal tip against the tissue at that point.

An embodiment of the disclosure is a system for sensing the electrical conductance of a material such as body tissue of a test subject. The system may sense the bio-conductivity of body tissue such as skin or some other tissue. The system may include an electrodermal probe/sensor for contacting a user's skin and reading the electrical conductance of the user's skin. The electrodermal probe may include one or more probe tips positioned on the electrodermal probe to contact a site of the user's skin. Each of the one or more probe tips may be independent from other of the one or more probe tips and may take independent measurements of the user's skin relative to other probe tips.

The measurements taken by the system can be assessed for determining a skin resistance measurement and/or a meridian conductivity measurement for the user. The meridian conductivity measurement may include a meridian stress assessment for measuring energy associated with acupuncture meridians. The measurements can be used in multiple healthcare practices such as bio resonance therapy, bio-energy regulatory techniques, biocybernetics medicine, computerized electrodermal screening, computerized electrodermal stress analysis, electrodermal testing, limbic stress assessment, meridian energy analysis, point testing, and others.

However, inaccuracies may occur by variability between technicians that apply different forces while taking measurements using bioelectric measurement systems. As different forces are applied by different technicians, contact between the conductive metal tip and the test point may vary, leading to inconsistent readings.

Automated bioelectric measurement systems have sought to limit variability that can occur during testing due to differences in forces applied by different technicians. These automated bioelectric measurement systems incorporate motors with a computerized control system. Although these automated systems reduce variability in sample measurements, the control system, the motor, motor components, and/or other elements in the system can become compromised by temperature as the motor functions and can become warm and even hot enough to affect the functioning of various components inside the system. For example, linear motors utilize powerful permanent magnets carefully configured with a coil to produce the necessary force to properly operate the automated bio conductance measurement system. When current flows through the coil it creates a magnetic field that reacts with the permanent magnets and creates a force that is relative with the current applied. The coil generates heat with current flow and the heat is congruent with the current applied. The more current that flows through the coil the more force is obtained and the more heat is produced. Permanent magnets will lose gauss force as temperature increases. Permanent magnets can lose their magnetism altogether if the temperature gets too high.

Cooling fans have been incorporated in some systems, but these fans can be inadequate during heavy use of a bioelectric measurement system and require the technician to suspend use of the bioelectric measurement system until it cools to an appropriate temperature. Additionally, fans incorporated into systems are noisy and run at high speeds at all times the probes are in use regardless of the internal temperature. In light of this deficiency, disclosed herein are systems, methods, and devices for detecting and controlling temperatures within an automated bioelectric measurement system by turning on or off a fan to cool the device, regulating fan speed for optimal cooling, adjusting motor output force to maintain consistent force during measurements based on device temperature, and/or shutting the device down to prevent system overheating to improve the accuracy of readings obtained.

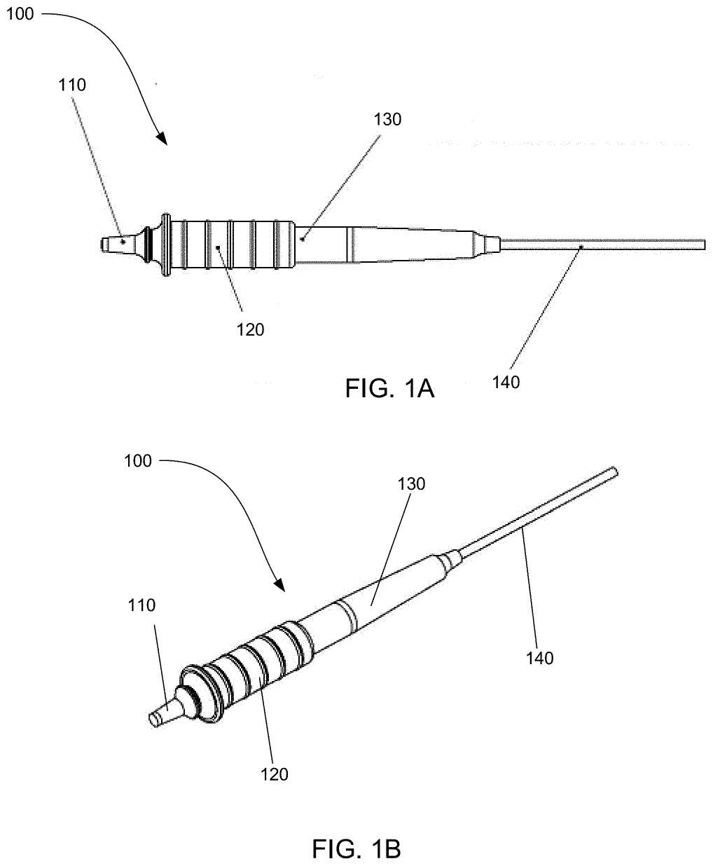

Now referring to the figures, FIGS. 1A and 1B illustrate an embodiment of a basic bioelectric measurement device 100. Bioelectric measurement device 100 may include a conductive metal tip 110 disposed at a distal end of bioelectric measurement device 100. A grip area 120 may be located adjacent to conductive metal tip 110 on a non-conductive probe body 130 of bioelectric measurement device 100. Internal parts, circuitry, wiring, and operating components of bioelectric measurement device 100 are enclosed in non-conductive probe body 130. Additionally, a cable 140 connects bioelectric measurement device 100 to an EAV system including a ground reference or hand mass portion of an EAV device. The cable 140 may be attached at a proximal end of non-conductive probe body 130 of bioelectric measurement device 100.

Bioelectric measurement device 100 is configured to measure the resistance of skin, meridian pathways in a body, and/or other materials or tissues. A technician holds bioelectric measurement device 100 in the grip area 120 while bioelectric testing is occurring. Conductive metal tip 110 can be applied to a body surface, such as skin of a test subject, to measure the resistance of skin or meridian pathway in the test subject. Non-conductive probe body 130 encases conductive elements and conductive metal tip 110, isolating the technician from the conductive metal tip 110 and limiting potential contamination in a measurement. Conductive metal tip 110 may be constructed of any suitably electrically conductive material such as copper, silver, gold, aluminum, zinc, nickel, brass, iron, steel, or other conductive material known to those skilled in the art.

In an embodiment, conductive metal tip 110 may be a single metal tip 110. Additionally, conductive metal tip 110 may include a plurality of individual metal tips. The plurality of metal tips may each take independent bioelectrical measurements. Conductive metal tip 110 may further be divided into one or more primary probe tips located at the center and one or more secondary probe tips that are positioned around the one or more primary metal tips located at the center. Conductive metal tips may be textured to help penetrate the insulation layer or cornified layer of the skin/tissue without puncturing it.

It will be appreciated that significant training and practice is required to develop the skill needed to apply conductive metal tip 110 to the skin consistently throughout the testing of meridian points across the body. Accuracy in conductivity measurements and assessments is paramount in ensuring proper treatment and/or diagnosis. Although grip area 120 and non-conductive body 130 help to prevent interference from the technician in the measurement, the variability in human input (e.g., variable applied pressure by different technician with conductive tip 110 against skin of a test subject) can produce inconsistent measurements.

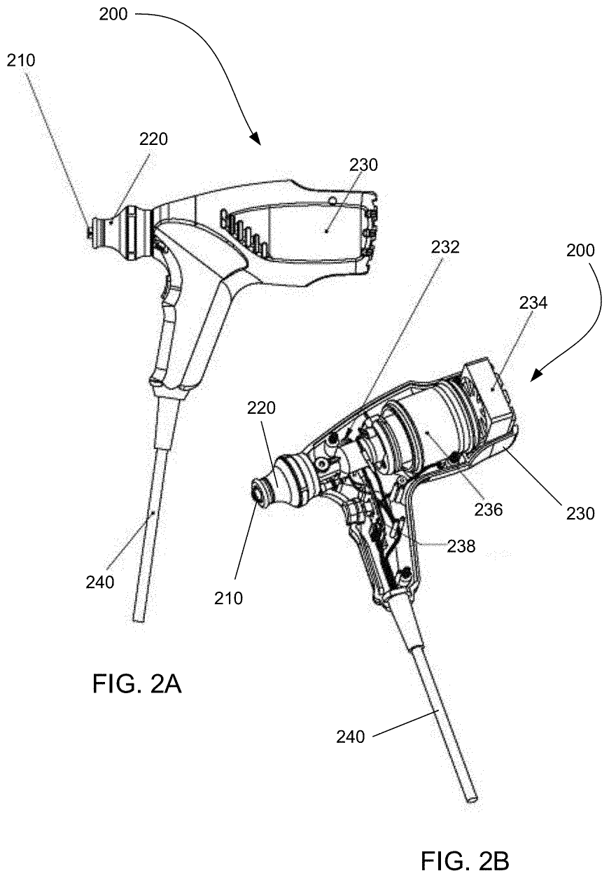

Referring now to FIG. 2A, a side view of an embodiment of an automated bioelectric measurement device 200 is illustrated. Automated bioelectric measurement device 200 is configured with conductive metal tip 210 located at a distal end of bioelectric measurement device 200. As shown, conductive metal tip 210 may be shielded by an isolating hood 220. A non-conductive body 230 may houses and electronically isolate internal parts, circuitry, wiring, and operating components of bioelectric measurement device 200. A cable 240 connects automated bioelectric measurement device 200 to an EAV system including a ground reference or hand mass portion of an EAV device (not illustrated) to allow electronic communication between the bioelectric measurement device 200 and the EAV device. As shown, cable 240 may be attached at an end or handle of non-conductive body 230 of bioelectric measurement device 200.

Automated bioelectric measurement device 200 is configured to measure the resistance of skin, meridian pathways in a body, or other materials or tissues, and can be applied to the skin surface to calculate the conductivity, meridian pathways, or other materials or tissues.

In an embodiment, conductive metal tip 210 may be a single metal tip 110. Additionally, conductive metal tip 110 may include a plurality of individual metal tips. The plurality of metal tips may each take independent bioelectrical measurements. Conductive metal tip 210 including the plurality of tips may be further divided into one or more primary conductive metal tips located at the center and one or more secondary conductive metal tips that are positioned around one or more primary conductive metal tips located at the center. Conductive metal tip 210 may be textured to help penetrate the insulation layer or cornified layer of the tissue without puncturing it. The one or more conductive metal tips may be constructed of any suitably electrically conductive material such as copper, silver, gold, aluminum, zinc, nickel, brass, iron, steel, or other material known to those skilled in the art.

It will be appreciated that isolating hood 220 provides increased shielding to conductive metal tip 210 during testing to limit contamination of measurements more effectively.

FIG. 2B illustrates automated bioelectric measurement device 200 with a portion of non-conductive probe body 230 removed to reveal interior components and systems. Internal components of automated bioelectric measurement device 200 include a thermal sensor 232, a fan 234 located at an opposite end of automated bioelectric measurement device 200 relative to conductive metal tip 210, a motor 236 connected to conductive metal tip 210 and located between fan 234 and conductive metal tip 210, and a printed circuit board 238 that electronically communicates with and controls thermal sensor 232, fan 234, and motor 236. Thermal sensor 232 may be a thermistor, or any other suitable thermal/temperature sensor

When a tissue or meridian point is being tested, isolating hood 220 is placed over the sample site on a body of a test subject. Automated bioelectric measurement device 200 then applies the force through motor 236 to extend conductive metal tip 210 to contact the sample site and automatically controls conductive metal tip 210, including applied force, through the entire point test with no technician interference. Automated bioelectric measurement device 200 then conducts the reading as conductive metal tip 210 is in contact with the test point.

It can be appreciated that motor 236 produces heat during use as it extends conductive metal tip 210 to contact the test site. It can be further appreciated that components of the system may be temperature sensitive such that production of heat by motor 236 throughout automated bioelectric measurement device 200 over time will decrease output force from motor 236 on conductive metal tip 210, resulting in compromised readings and false assessments because of the variability in output forces. Heat produced by motor 236 during use may additionally compromise logic systems in printed circuit board 238 that control automated bioelectric measurement device 200.

The fan may be located at an opposite end of automated bioelectric measurement device 200 relative to conductive metal tip 210 to produce air flow that can cool motor 236 to prevent system overheating. Current bioelectric measurement systems containing fans do not include mechanisms to control fan activity when cooling is needed or when cooling is not needed. Additionally, the fans run at high speeds at all times regardless of system temperatures, producing excessive noise and possible excessive interference with testing.

In light of these deficiencies, in an exemplary embodiment shown in FIG. 2B, thermal sensor 232 is located on printed circuit board 238 to measure internal temperatures in the bioelectric measurement device 200. Thermal sensor 232 may be placed in any optimal location within the bioelectric measurement system 200 to acquire the most significant internal temperatures during use or temperatures of any particular component within the system. Thermal sensor 232 may control a system in printed circuit board 238 that turns on fan 234 when temperatures are warm or hot and cooling is needed, or turn off the fan 234 when temperatures reach a desired level and cooling is no longer required. The temperature input from thermal sensor 232 may variably control the speed of fan 234 to provide adequate air flow to cool motor 236 and maintain an appropriate heat level for continued use of the bioelectric measurement system. The thermal sensor may be a single thermal sensor 232. Alternatively, the thermal sensor may include a plurality of thermal sensors distributed to different positions throughout the bioelectric measurement device 200. Each of a plurality of thermal sensors may take a temperature measurement that is independent of temperature measurements taken by other of the plurality of thermal sensors.

Maintenance of appropriate temperatures within bioelectric measurement device 200 and motor 236 improves operation of motor 236 and various components of motor 236 in order to decrease fluctuations in output forces on conductive metal tip 210 and damage to printed circuit board 238 to improve consistency and accuracy in readings. For example, as temperatures around motor 236 increase, gauss force within internal components of motor 236 may decrease. A decrease in gauss force within motor 236 decreases the applied output force from motor 236 relative to its input, resulting in variability in the output force on conductive metal tip 210.

Additionally, motor 236 may be controlled through circuitry on printed circuit board 238 to modulate, adjust, or compensate the force applied by motor 236 based on temperature measurements from thermal sensor 232. For example, in the event of high temperatures in bioelectric measurement device 200 during heavy use, maximum airflow from fan 234 may be inadequate to cool the motor to a desired level leading to variability in applied force by motor 236. In such a case, motor 236 may be modulated/adjusted to produce an output force within an acceptable range, based on temperature measurements from thermal sensor 232, to maintain consistency in bioelectric readings.

In the event that highest airflow from fan 234 and motor 236 compensation is inadequate, measurements from thermal sensor 232 and communication from circuitry on printed circuit board 238 may alert the technician that the temperature of bioelectric measurement device 200 has exceeded an acceptable range and shut down bioelectric measurement device 200 until temperature levels fall within an acceptable range. After the temperature levels fall within the acceptable range, bioelectric measurement device 200 may be allowed to turn back on and operate again. The alert to the technician may be a visual alert (e.g., a message displayed on a display or an indicator light turning on or flashing in a certain pattern or color), an audio alert (e.g., a sound/message emitted from a speaker), a tactile alert (e.g., vibration or other alert that is felt by a user in any pattern), or any other alert method known in the art.

A range of acceptable temperature measurements for internal temperatures in the bioelectric measurement system may be stored within memory on printed circuit board 238. The range of acceptable temperatures may be stored in and retrieved from a database, may be stored in an EAV, computer, or similar devices connected to the bioelectric measurement system, and may be stored in and received from a computing device over a network or other communication source, and so forth.

Printed circuit board 238 includes one or more processors/controllers that are configurable to execute instructions stored in non-transitory computer readable storage media. The instructions may include receiving a temperature measurement from thermal sensor 232 located within the non-conductive probe body 230 of the bioelectric measurement system. The instructions include assessing the temperature within the bioelectric measurement system, comparing the measured temperature to an acceptable range of standard temperatures retrieved from memory, determining whether cooling is required and then turning fan 234 on or off and adjusting the fan speed to provide the appropriate cooling required to maintain optimal temperatures within bioelectric measurement device 200.

The temperature measurements may be acquired from a plurality of thermal sensors 232 located within the non-conductive probe body 230 and an average temperature within the bioelectric measurement device 200 may be calculated and used as the thermal input to assess and regulate fan 234 activity.

The temperature measurement recorded by thermal sensor 232 may be transmitted through cable 240 to an EAV or similar device. One or more processors/controllers in the EAV or similar device performs the calculations to compare temperature measurements to the acceptable range and calculates the required fan activity and speed to provide optimal cooling. The adjustment is then transmitted back through cable 240 to probe printed circuit board 238 to regulate fan 234. It is also recognized that communication and data can be transmitted by wireless means such as Bluetooth.

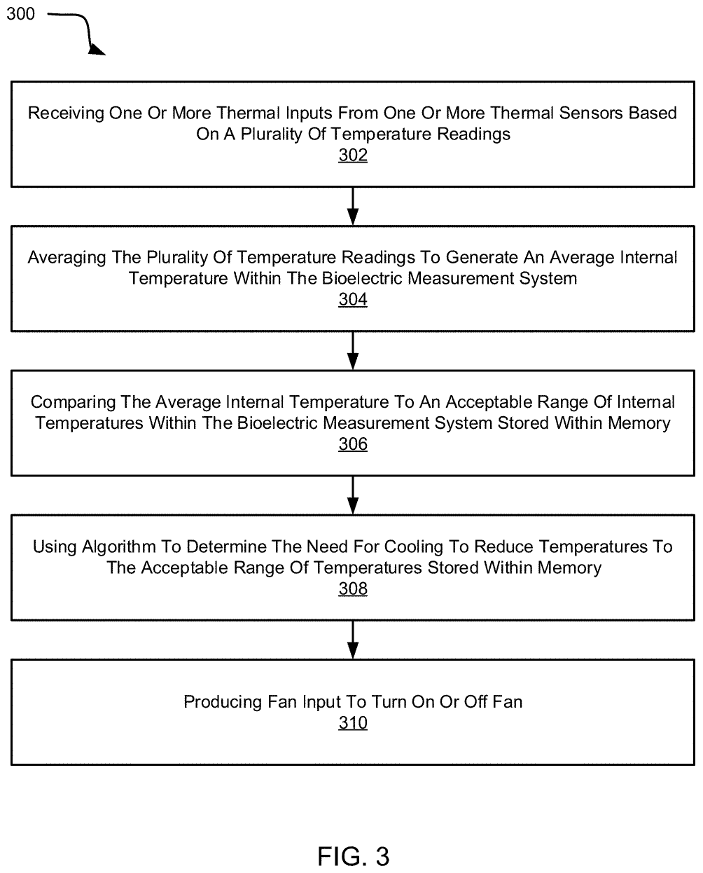

FIG. 3 is a schematic flow chart diagram of a method 300 for measuring internal temperature within a bioelectric measurement system and regulating the turning on or off a fan for cooling of internal components. The method 300 may be performed by any suitable computing device, such as one or more processors/controllers of printed circuit board 238 in electrical communication with a bioelectric measurement device 200, or in a computing device connected to bioelectric measurement device 200.

As step 302 of method 300, one or more thermal sensors measure the temperature within bioelectric measurement system and produces a thermal input, after which the thermal inputs are transmitted from the one or more thermal sensors to one or more processors/controllers in the computing device. In step 304, the computing device calculates an average internal temperature for the bioelectric measurement system based on the thermal inputs received from the one or more thermal sensors. In step 306 of method 300, the computing device compares the average temperature to a range of acceptable temperature readings stored within memory. In step 308 of method 300, a computing device uses an algorithm to then determine if the average internal temperature in the bioelectric measurement system is above or within the acceptable range of temperatures stored in memory and determines a need for cooling to reduce temperatures within the system. In step 310, the computing device produces a fan input that will either turn on or off the fan as needed to provide cooling to the bioelectric measurement system. The method may then repeat and continue throughout use of the bioelectric measurement system to monitor and respond to changes in temperature during use.

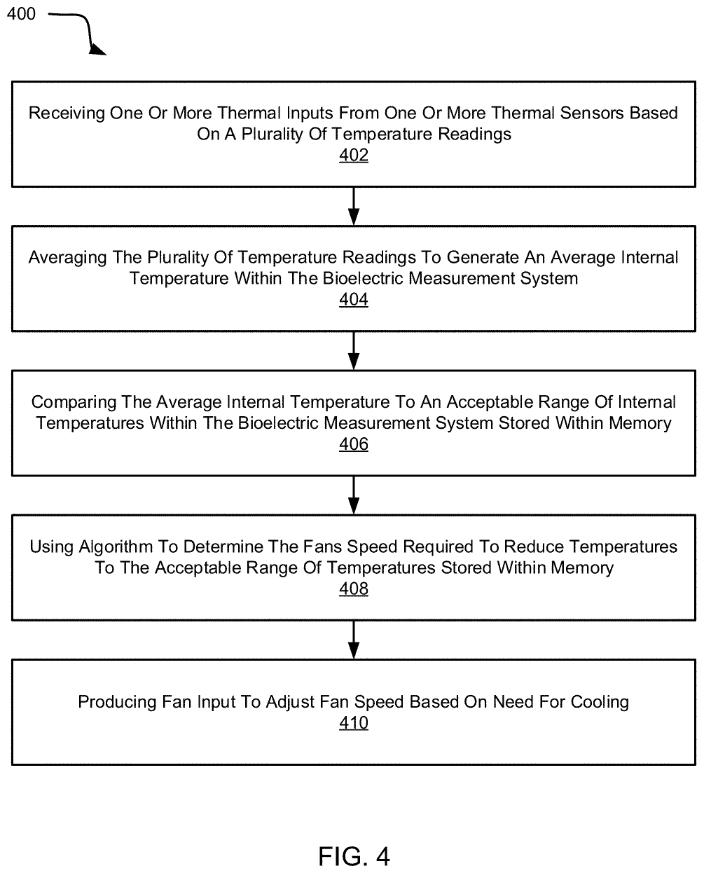

FIG. 4 is a schematic flow chart diagram of a method 400 for measuring temperatures within a bioelectric measurement system and regulating fan speed to provide optimal cooling with a bioelectric measurement system. The method 400 may be performed by any suitable computing device, such as one or more processors/controllers of printed circuit board 238 in electrical communication with a bioelectric measurement device 200, or in a computing device connected to bioelectric measurement device 200.

As step 402 of method 400, one or more thermal sensors measure the temperature within bioelectric measurement system and produces a thermal input, after which the thermal inputs are transmitted from the one or more thermal sensors to one or more processors/controllers in the computing device. In step 404, the computing device calculates an average internal temperature for the bioelectric measurement system based on the thermal inputs received from the one or more thermal sensors. In step 406 of method 400, the computing device compares the average temperature to a range of acceptable temperature readings stored within memory. In step 408 of method 400, a computing device then uses an algorithm to determine the amount of cooling and/or fan speeds required to decrease temperatures to fall within an acceptable range. In step 410, The computing device controls the fan and produces fan input to then increase or decrease fan speed as needed to provide cooling. The method may then repeat and continue throughout use of the bioelectric measurement system to monitor and respond to changes in temperature during use.

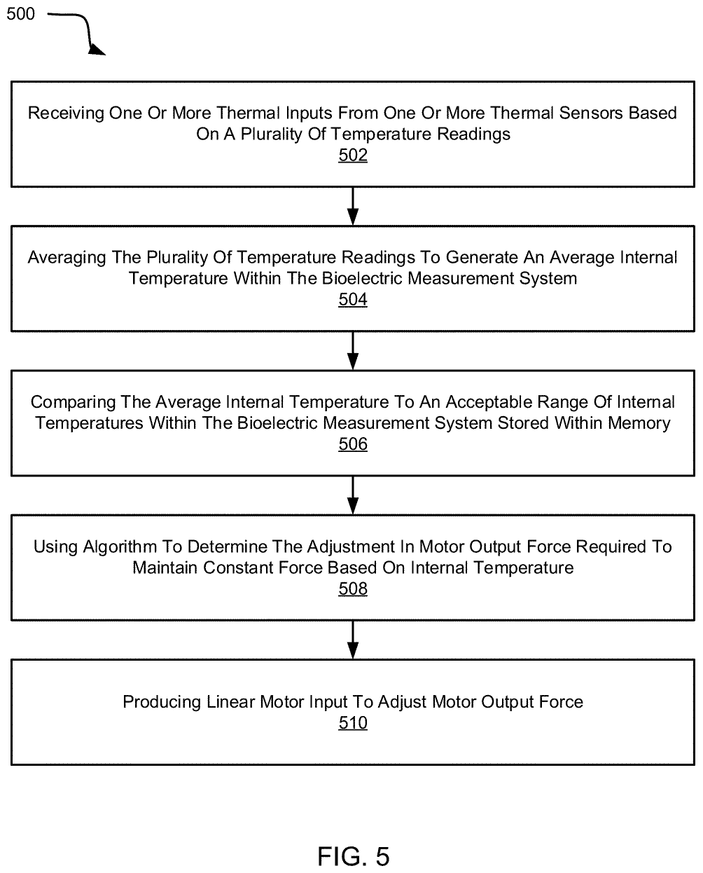

FIG. 5 is a schematic flow chart diagram of a method 500 for measuring temperatures within a bioelectric measurement system and adjusting output force in motor to compensate for decreases in forces resulting from variable temperatures within bioelectric measurement device 200. The method 500 may be performed by any suitable computing device, such as one or more processors/controllers of printed circuit board 238 in electrical communication with a bioelectric measurement device 200, or in a computing device connected to bioelectric measurement device 200.

As step 502 of method 500, one or more thermal sensors measure the temperature within bioelectric measurement system and produces a thermal input, after which the thermal inputs are transmitted from the one or more thermal sensors to one or more processors/controllers in the computing device. In step 504, the computing device calculates an average internal temperature for the bioelectric measurement system based on the thermal inputs received from the one or more thermal sensors. In step 506 of method 500, the computing device compares the average temperature to a range of acceptable temperature readings stored within memory. In step 508 of method 500, the computing device then uses an algorithm 508 to calculate an increase or decrease in output force for the motor in response to changes in temperature, in order to maintain constant or desired force of the motor. The method may then repeat continue as the computing device produces a motor input to adjust the level of output force for the motor to maintain constant and/or desired force throughout use to improve consistency and accuracy of measurements.

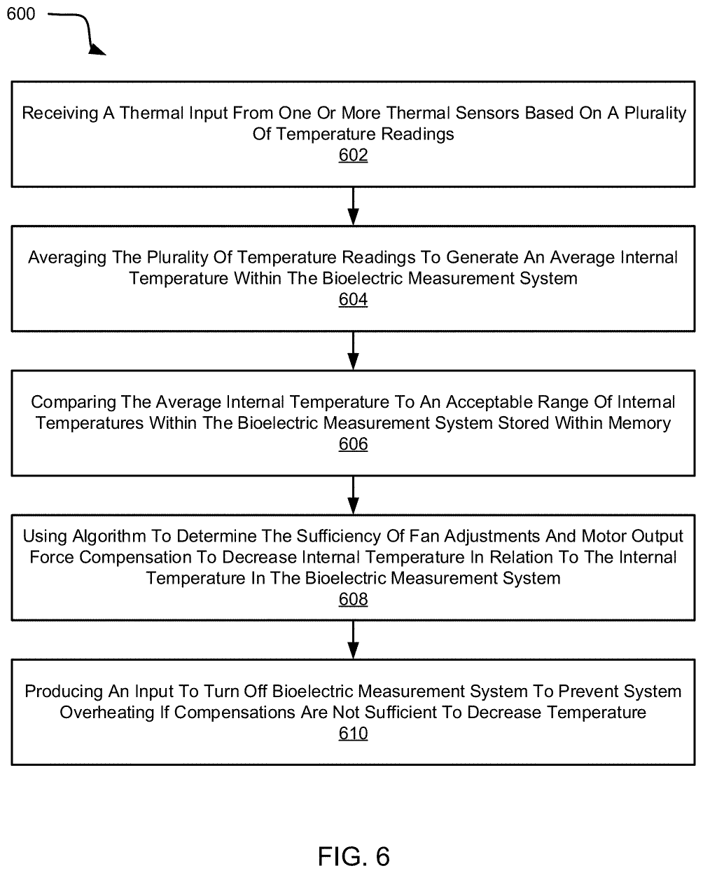

FIG. 6 is a schematic flow chart diagram of a method 600 for measuring temperatures within a bioelectric measurement system and shutting down bioelectric measurement device 200 to prevent system overheating. The method 600 may be performed by any suitable computing device such as one or more processors/controllers of printed circuit board 238 in electrical communication with a bioelectric measurement device 200, or in a computing device connected to bioelectric measurement device 200.

As step 602 of method 600, one or more thermal sensors measure the temperature within bioelectric measurement system and produce a thermal input, after which the thermal inputs are transmitted from the one or more thermal sensors to one or more processors/controllers in the computing device. In step 604, computing device calculates an average internal temperature for the bioelectric measurement system based on the thermal inputs received. In step 606 of method 600, the computing device compares the average temperature to a range of acceptable temperature readings stored within memory 606. In step 608, method 600 continues and a computing device uses an algorithm 608 to evaluate and determine the sufficiency of fan adjustments and motor output force adjustment and compensation to decrease the internal temperature. The algorithm determines the sufficiency of adjustments made to decrease the internal temperature in the bioelectric measurement system and the current internal temperature, and, in step 610, will produce an input to shut off the bioelectric measurement system if adjustments are insufficient to decrease internal temperatures in the bioelectric measurement system to an acceptable range. Once temperature decreases to acceptable levels (e.g., below or within a predefined range of temperatures) the bioelectric measurement system may be allowed to restart and operate again.

As discussed above with relation to FIGS. 3-6, thermal sensor 232 may be a plurality of thermal sensors which each take one or more temperature measurements. In methods 300-600, the plurality of temperature measurements from the plurality of thermal sensors may be averaged to determine the average internal temperature of bioelectric measurement system and following calculations/adjustments to the system are made based on the average internal temperature. Alternatively, a single thermal sensor 232 may be used and relevant calculations and adjustments may be made based on the single temperature measurement from a single thermal sensor. Additionally, multiple temperature measurements taken over time from the single thermal sensor may also be used for relevant calculations and adjustments.

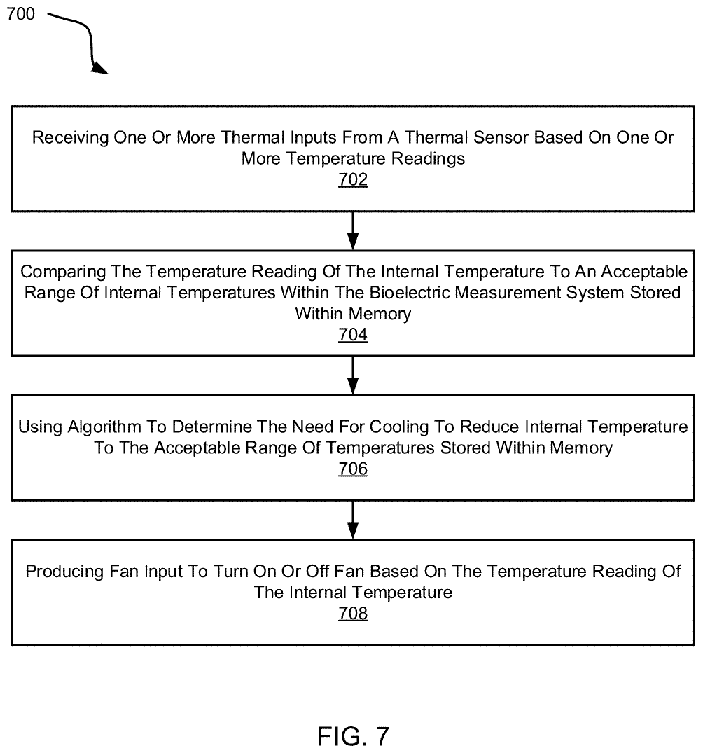

For example, FIG. 7 is a schematic flow chart diagram of a method 700 for measuring internal temperature within a bioelectric measurement system and regulating the turning on or off a fan for cooling of internal components. Method 700 is similar to method 300 but uses one thermal sensor as opposed to multiple thermal sensors. The method 700 may be performed by any suitable computing device, such as one or more processors/controllers of printed circuit board 238 in electrical communication with a bioelectric measurement device 200, or in a computing device connected to bioelectric measurement device 200.

As step 702 of method 700, a thermal sensor measures the temperature within bioelectric measurement system and produces a thermal input, after which the thermal input is transmitted from the thermal sensor to one or more processors/controllers in the computing device. In step 704 of method 700, the computing device compares the temperature to a range of acceptable temperature readings stored within memory. In step 706 of method 700, a computing device uses an algorithm to then determine if the internal temperature in the bioelectric measurement system is above or within the acceptable range of temperatures stored in memory and determines a need for cooling to reduce temperatures within the system. In step 708, the computing device produces a fan input that will either turn on or off the fan as needed to provide cooling to the bioelectric measurement system. The method may then repeat and continue throughout use of the bioelectric measurement system to monitor and respond to changes in temperature during use.

Although method 700 is illustrated in relation to similar method 300, it is to be understood that method 700 (e.g., using a single thermal sensor) may also be applied to methods 400-600 as well. In other words, any of methods 300-600 may utilize a single thermal sensor instead of using multiple thermal sensors and averaging the multiple values together. Additionally, an average of a plurality of temperature values taken from a single sensor over time may also be used to perform any of methods 300-600, or similarly, the plurality of thermal sensors may each either take one temperature measurement, a plurality of temperature measurements over time, and an average may be calculated based on a single measurement from each thermal sensor or multiple temperature measurements from each thermal sensor.

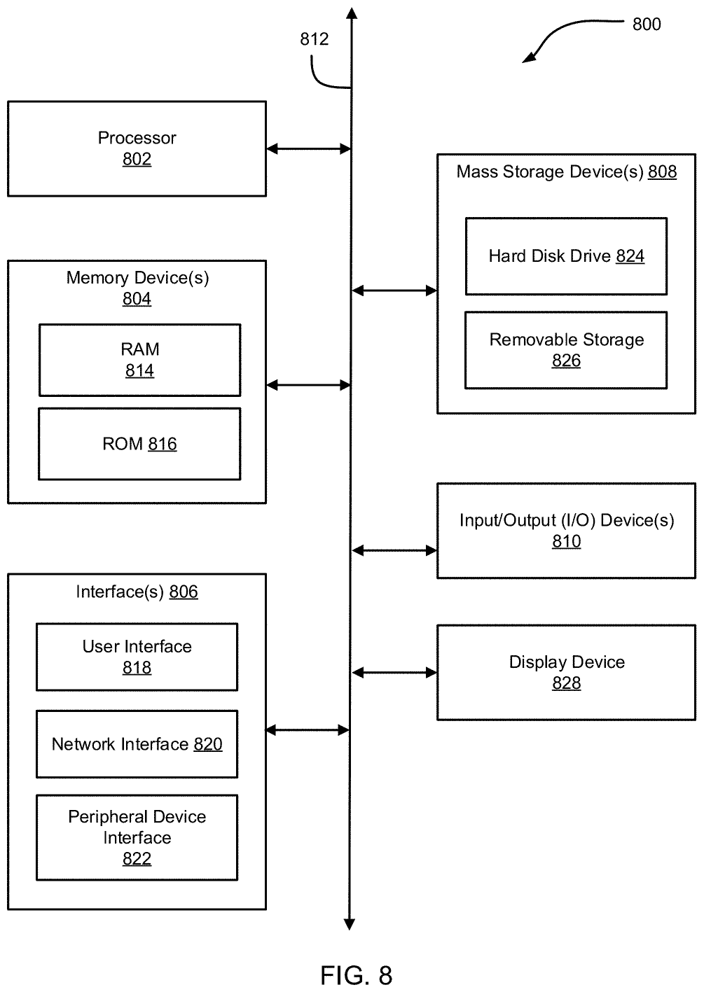

FIG. 8 is a block diagram illustrating an example computing device 800. Computing device 800 may be used to perform various procedures, such as those discussed herein. Computing device 800 can function as a server, a client, or any other computing entity such as the printed circuit board 238 in communication with the bioelectric measurement device 200. Computing device can perform various monitoring functions as discussed herein, and can execute one or more application programs, such as the application programs described herein. Computing device 800 can be any of a wide variety of computing devices, such as probe printed circuit board 238, a desktop computer, a notebook computer, a server computer, a handheld computer, tablet computer and the like.

Computing device 800 may include one or more processor(s) 802, one or more memory device(s) 804, one or more interface(s) 806, one or more mass storage device(s) 808, one or more Input/Output (I/O) device(s) 810, and a display device 828 all of which are coupled to a bus 812. Processor(s) 802 include one or more processors/controllers or controllers that execute instructions stored in memory device(s) 804 and/or mass storage device(s) 808. Processor(s) 802 may also include various types of computer-readable media, such as cache memory.

Memory device(s) 804 include various computer-readable media, such as volatile memory (e.g., random access memory (RAM) 814) and/or nonvolatile memory (e.g., read-only memory (ROM) 816). Memory device(s) 804 may also include rewritable ROM, such as Flash memory.

Mass storage device(s) 808 include various computer readable media, such as magnetic tapes, magnetic disks, optical disks, solid-state memory (e.g., Flash memory), and so forth. As shown in FIG. 7, a particular mass storage device is a hard disk drive 824. Various drives may also be included in mass storage device(s) 808 to enable measurement from and/or writing to the various computer readable media. Mass storage device(s) 808 include removable media 826 and/or non-removable media.

I/O device(s) 810 include various devices that allow data and/or other information to be input to or retrieved from computing device 800. Example I/O device(s) 810 include cursor control devices, keyboards, keypads, microphones, monitors or other display devices, speakers, printers, network interface cards, modems, lenses, CCDs or other image capture devices, and the like.

Display device 828 includes any type of device capable of displaying information to one or more users of computing device 800. Examples of display device 828 include a monitor, display terminal, video projection device, and the like.

Interface(s) 806 include various interfaces that allow computing device 800 to interact with other systems, devices, or computing environments. Example interface(s) 806 may include any number of different network interfaces 820, such as interfaces to local area networks (LANs), wide area networks (WANs), wireless networks, and the Internet. Other interface(s) include user interface 818 and peripheral device interface 822. The interface(s) 806 may also include one or more user interface elements 818. The interface(s) 806 may also include one or more peripheral interfaces such as interfaces for printers, pointing devices (mice, track pad, or any suitable user interface now known to those of ordinary skill in the field, or later discovered), keyboards, and the like.

Bus 812 allows processor(s) 802, memory device(s) 804, interface(s) 806, mass storage device(s) 808, and I/O device(s) 810 to communicate with one another, as well as other devices or components coupled to bus 812. Bus 812 represents one or more of several types of bus structures, such as a system bus, PCI bus, IEEE 1394 bus, USB bus, and so forth.

For purposes of illustration, programs and other executable program components are shown herein as discrete blocks, although it is understood that such programs and components may reside at various times in different storage components of computing device 800 and are executed by processor(s) 802. Alternatively, the systems and procedures described herein can be implemented in hardware, or a combination of hardware, software, and/or firmware. For example, one or more application specific integrated circuits (ASICs) can be programmed to carry out one or more of the systems and procedures described herein.

Implementations of the disclosure may comprise or utilize a special purpose or general-purpose computer including computer hardware, such as, for example, one or more processors/controllers and system memory, as discussed in greater detail below. Implementations within the scope of the disclosure also include physical and other computer-readable media for carrying or storing computer-executable instructions and/or data structures. Such computer-readable media can be any available media that can be accessed by a general purpose or special purpose computer system. Computer-readable media that store computer-executable instructions are computer storage media (devices). Computer-readable media that carry computer-executable instructions are transmission media. Thus, by way of example, and not limitation, implementations of the disclosure can comprise at least two distinctly different kinds of computer-readable media: computer storage media (devices) and transmission media.

Computer storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmission media can include a network and/or data links, which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to computer storage media devices or vice versa. For example, computer-executable instructions or data structures received over a network or data link can be buffered in RAM 814 within a network interface module 820 (e.g., a “NIC”), and then eventually transferred to computer system RAM 814 and/or to less volatile computer storage media (devices) at a computer system. RAM 814 can also include solid state drives (SSDs or PCIx based real time memory tiered storage, such as FusionIO). Thus, it should be understood that computer storage media devices can be included in computer system components that also (or even primarily) utilize transmission media.

Computer-executable instructions comprise, for example, instructions and data which, when executed at a processor, cause a general-purpose computer, special purpose computer, or special purpose processing device to perform a certain function or group of functions. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, various storage devices, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Implementations of the disclosure can also be used in cloud computing environments. In this description and the following claims, “cloud computing” is defined as a model for enabling ubiquitous, convenient, on-demand network access to a shared pool of configurable computing resources (e.g., networks, servers, storage, applications, and services) that can be rapidly provisioned via virtualization and released with minimal management effort or service provider interaction, and then scaled accordingly. A cloud model can be composed of various characteristics (e.g., on-demand self-service, broad network access, resource pooling, rapid elasticity, measured service, or any suitable characteristic now known to those of ordinary skill in the field, or later discovered), service models (e.g., Software as a Service (SaaS), Platform as a Service (PaaS), Infrastructure as a Service (IaaS)), and deployment models (e.g., private cloud, community cloud, public cloud, hybrid cloud, or any suitable service type model now known to those of ordinary skill in the field, or later discovered). Databases and servers described with respect to the disclosure can be included in a cloud model.

Further, where appropriate, functions described herein can be performed in one or more of: hardware, software, firmware, digital components, or analog components. For example, one or more application specific integrated circuits (ASICs) can be programmed to carry out one or more of the systems and procedures described herein. Certain terms are used throughout the following description and Claims to refer to particular system components. As one skilled in the art will appreciate, components may be referred to by different names. This document does not intend to distinguish between components that differ in name, but not function.

### EXAMPLES

The following examples pertain to further embodiments.

Example 1 is a bioelectric measurement system, wherein the bioelectric measurement system comprises a non-conductive housing, a conductive metal tip connected to a motor, a fan located distal relative to the conductive metal tip, a printed circuit board that communicates with the motor and fan, and a thermal sensor located in the non-conductive housing, wherein the motor applies controlled force to the conductive metal tip to determine skin conductivity, and wherein the thermal sensor measures internal temperatures in the bioelectric measurement system and conveys a temperature measurement to the printed circuit board to send instructions to the fan.

Example 2 is a device as in Example 1, wherein the instructions sent to the fan operate to turn the fan on or off depending upon the internal temperatures of the bioelectric measurement system.

Example 3 is a device as in any of Examples 1-2, wherein conductive metal tip is rigidly connected to the motor and wherein the motor includes a controller to regulate output force.

Example 4 is a device as in any of Examples 1-3, wherein the bioelectric measurement system comprises a plurality of conductive metal tips for sensing resistivity of tissue, wherein each of the plurality of conductive metal tips is independent and makes an independent measurement of resistivity of a tissue.

Example 5 is a device as in any of Examples 1-4, wherein the non-conductive body incorporates an isolating hood that surrounds the conductive metal top and isolates the technician from the probe.

Example 6 is a device as in any of Examples 1-5, wherein the thermal sensor is located on the printed circuit board.

Example 7 is a device as in any of Examples 1-6, wherein the thermal sensor is located in close proximity to the motor to optimally measure temperature output from the motor.

Example 8 is a device as in any of Examples 1-7, wherein the bioelectric measurement system includes a plurality of thermal sensors disposed throughout the non-conductive housing to optimally measure internal temperatures in the bioelectric measurement system.

Example 9 is a device as in any of Examples 1-8, wherein the thermal sensor produces a thermal input based on the temperature measurement that is electronically conveyed to a processor on the printed circuit board.

Example 10 is a device as in any of Examples 1-9, wherein the thermal sensor produces a thermal input based off of the temperature measurement that is electronically conveyed to a processor on a computing device through a cable or wireless transmission.

Example 11 is a device as in any of Examples 1-10, wherein a processor receives a plurality of independent thermal inputs based on temperature readings from a plurality of thermal sensors and calculates the average temperature within the bioelectric measurement system based on the thermal inputs received from the one or more thermal sensors.

Example 12 is a device as in any of Examples 1-11, wherein a processor compares the average internal temperature to an acceptable range of temperatures stored within memory and turns on the fan if cooling is need and turns off the fan if cooling is not needing to reduce internal temperatures to fall within the acceptable temperature range.

Example 13 is a device as in any of Examples 1-12, wherein the processor uses an algorithm that compares the average internal temperature to an acceptable range of temperatures stored within memory and variably adjusts fan speed according to the magnitude of cooling required to reduce internal temperatures to fall within the acceptable temperature range.

Example 14 is a device as in any of Examples 1-13, wherein the processor uses an algorithm that compares the average internal temperature to an acceptable range of temperatures stored within memory and calculates differences in motor output force based on internal temperatures and produces a motor input to adjust motor output force to maintain the proper motor output force during use.

Example 15 is a device as in any of Examples 1-14, wherein the processor uses an algorithm that compares the average internal temperature to an acceptable range of temperatures stored within memory, assesses the efficiency of fan and motor adjustments to regulate internal temperatures and determines whether to turn the device off to prevent device overheating.

Example 16 is a method. The method includes measuring the temperature within the bioelectric measurement system using one or more thermal sensors.

Example 17 is a method as in Example 16, wherein the method includes producing a thermal input based on the temperature reading measured by the one or more thermal sensors and electronically conveying the thermal input to the printed circuit board.

Example 18 is a method as in Examples 16-17. The method includes producing a thermal input based on the temperature reading measured by the one or more thermal sensors and electronically conveying the thermal input to a processor on a computing device through a cable or wireless transmission.

Example 19 is a method as in Examples 16-18. The method includes receiving one or more thermal inputs from the one or more thermal sensor and calculating the average internal temperature based on the thermal inputs received using a processor.

Example 20 is a method as in Examples 16-19. The method includes calculating an average temperature measurement and comparing the average temperature to an acceptable temperature range for the bioelectric measurement system recalled from memory using a processor.

Example 21 is a method as in Examples 16-20, wherein after receiving the one or more thermal inputs, calculating an average internal temperature and comparing the average temperature to the acceptable temperature range, the processor uses an algorithm to determine if the fan needs to be turned on or off to reduce temperatures within in the bioelectric measurement system and produces a fan input to turn on or off the fan.

Example 22 is a method as in any of Examples 16-21, wherein after receiving the one or more thermal inputs, calculating an average internal temperature and comparing the average temperature to the acceptable temperature range, the processor uses an algorithm to determine the fan speed required to reduce temperatures within the bioelectric measurement system to fall within the acceptable temperature range and produces a fan input to adjust fan speed.

Example 23 is a method as in any of Examples 16-22, wherein after receiving the one or more thermal inputs, calculating an average internal temperature and comparing the average temperature to the acceptable temperature range, the processor uses an algorithm to calculate variability in motor output force based on the average internal temperature and produces a motor input to adjust motor output force to maintain consistent and proper force application despite temperature changes within the bioelectric measurement system.

Example 24 is a method as in any of Examples 16-23, wherein after receiving the one or more thermal inputs, calculating an average internal temperature and comparing the average temperature to the acceptable temperature range, the processor uses an algorithm to assess the internal temperature in the bioelectric measurement system and the efficiency of fan adjustments and motor compensation, and determines whether to turn off the device to prevent system overheating and damage.

Example 25 is non-transitory computer readable storage media storing instructions to be executed by one or more processors/controllers, the instructions comprising: receiving one or more thermal inputs from the one or more thermal sensors located in the non-conductive housing of a bioelectric measurement system; and using an algorithm to calculate the average temperature in the bioelectric measurement system based on thermal inputs received.

Example 26 is non-transitory computer readable storage media as in Example 25, the instructions comprising: receiving one or more thermal inputs from the one or more thermal sensors located in the non-conductive housing of a bioelectric measurement system and comparing the average temperature to an acceptable temperature range within the bioelectric measurement system stored in memory.

Example 27 is non-transitory computer readable storage media as any of Examples 25-26, the instructions comprising: receiving one or more thermal inputs from the one or more thermal sensors located in the non-conductive housing of a bioelectric measurement system; calculating the average temperature and comparing the average internal temperature to an acceptable temperature range stored within memory; and using an algorithm to determine the need for the fan to be turned on or off based on the need to decrease the average internal temperature.

Example 28 is non-transitory computer readable storage media as any of Examples 25-27, the instructions comprising: receiving one or more thermal inputs from the one or more thermal sensors located in the non-conductive housing of a bioelectric measurement system; calculating the average temperature and comparing the average internal temperature to an acceptable temperature range stored within memory; and using an algorithm to determine the fan speed required to provide the amount of cooling required to reduce the average internal temperature to fall within the acceptable temperature range.

Example 29 is non-transitory computer readable storage media as in any of Examples 25-28, wherein the instructions are such that: receiving one or more thermal inputs from the one or more thermal sensors located in the non-conductive housing of a bioelectric measurement system; calculating the average temperature and comparing the average internal temperature to an acceptable temperature range stored within memory; and using an algorithm to calculate the variability in output force based on internal temperature and determining the required adjustment to output force in the motor to maintain consistent force throughout use.

Example 30 is non-transitory computer readable storage media as in any of Examples 25-29, wherein the instructions are such that: receiving one or more thermal inputs from the one or more thermal sensors located in the non-conductive housing of a bioelectric measurement system; calculating the average temperature and comparing the average internal temperature to an acceptable temperature range stored within memory; and using an algorithm to calculate the efficiency of adjustments to the fan and motor in relation to the average internal temperature in the bioelectric measurement system and determining whether to shut off the bioelectric measurement system to prevent system overheating.

Example 31 is a bioelectric measurement device. The bioelectric measurement device may include a housing, a conductive tip disposed at a distal end of the housing, a motor within the housing that applies a motor output force to the conductive tip, a thermal sensor that takes a temperature measurement of an inside of the housing, a fan that cools the inside of the housing including the motor, and a controller that controls the fan based on the temperature measurement from the thermal sensor.

Example 32 is a device as in Example 31, wherein the controller turns the fan on and off based on the temperature measurement.

Example 33 is a device as in any of Examples 31-32, wherein the controller turns the fan on if the temperature is higher than a predefined temperature range and turns the fan off if the temperature measurement is within or lower than the predefined temperature range.

Example 34 is a device as in any of Examples 31-33, wherein the controller variably controls the fan speed based on based on where the temperature measurement falls relative to the predefined temperature range.

Example 35 is a device as in any of Examples 31-34, wherein the controller controls the motor such that the motor output force applied to the conductive tip is automated; wherein the motor output force is variable, and the controller adjusts the motor output force based on the temperature measurement.

Example 36 is a device as in any of Examples 31-35, wherein, when the fan as controlled does not keep the temperature measurement within a predefined temperature range, the controller shuts down the bioelectric measurement device.

Example 37 is a device as in any of Examples 31-36, wherein, when the fan, as controlled, does not keep the temperature measurement within a predefined temperature range, and the motor, as controlled, does not maintain a desired motor output force, the controller shuts down the bioelectric measurement device.

Example 38 is a device as in any of Examples 31-37, wherein the controller alerts the user about the bioelectric measurement device shutting down.

Example 39 is a device as in any of Examples 31-38, wherein the thermal sensor comprises a plurality of thermal sensors disposed at a plurality of locations in the housing to obtain one or more temperature values from each of the plurality of thermal sensors.

Example 40 is a device as in any of Examples 31-39, wherein the temperature measurement is an average of the temperature values obtained from the plurality of thermal sensors.

Example 41 is a device as in any of Examples 31-40, wherein the housing is a non-conductive housing comprising: a non-conductive body; and an isolating hood surrounding the conductive tip to isolate a technician from other elements of the bioelectric measurement device.

Example 42 is a method for taking bioelectric measurements with a bioelectric measurement device comprising a housing, a conductive tip disposed at a distal end of the housing, a motor within the housing that applies a motor output force to the conductive tip, a thermal sensor that takes a temperature measurement of an inside of the housing, a fan that cools the inside of the housing including the motor, and a controller that controls the fan based on the temperature measurement from the thermal sensor. The method may include: receiving, by the controller, a temperature measurement from the thermal sensor; comparing the temperature measurement to a predefined temperature range; and controlling the fan based on the temperature measurement.

Example 43 is a method as in Example 42, wherein controlling the fan based on the temperature measurement comprises turning the fan on or off based on the temperature measurement relative to the predefined temperature range.

Example 44 is a method as in any of Examples 42-43, wherein controlling the fan based on the temperature measurement comprises turning the fan on if the temperature is higher than a predefined temperature range and turns the fan off if the temperature measurement is within or lower than the predefined temperature range.

Example 45 is a method as in any of Examples 42-44, wherein controlling the fan based on the temperature measurement comprises variably controlling the fan speed based on where the temperature measurement falls relative to the predefined temperature range.

Example 46 is a method as in any of Examples 42-45, wherein the controller controls the motor such that the motor output force applied to the conductive tip is automated and variable, the method further comprising: adjusting the motor output force based on the temperature measurement.

Example 47 is a method as in any of Examples 42-46, further comprising: shutting down the bioelectric measurement device when the fan, as controlled, does not keep the temperature measurement within a predefined temperature range.

Example 48 is a method as in any of Examples 42-47, further comprising: shutting down the bioelectric measurement device when the fan, as controlled, does not keep the temperature measurement within a predefined temperature range, and the motor, as controlled, does not maintain a desired motor output force.

Example 49 is a method as in any of Examples 42-48, further comprising: alerting the user about the bioelectric measurement device shutting down.

Example 50 is a method as in any of Examples 42-49, wherein the thermal sensor comprises a plurality of thermal sensors disposed at a plurality of locations in the housing to obtain one or more temperature values from each of the plurality of thermal sensors, and the method further comprises: calculating an average of the temperature values obtained from the plurality of thermal sensors and using the average as the temperature measurement.

The foregoing description has been presented for the purposes of illustration and description. It is not intended to be exhaustive or to limit the disclosure to the precise form disclosed. Many modifications and variations are possible in light of the above teaching. Further, it should be noted that any or all of the aforementioned alternate implementations may be used in any combination desired to form additional hybrid implementations of the disclosure.

Further, although specific implementations of the disclosure have been described and illustrated, the disclosure is not to be limited to the specific forms or arrangements of parts so described and illustrated. The scope of the disclosure is to be defined by the claims appended hereto, any future claims submitted here and in different applications, and their equivalents.

What is claimed is:

1. A bioelectric measurement device comprising:
a housing;
a conductive tip disposed at a distal end of the housing;
a motor within the housing that applies a motor output force to the conductive tip;
a thermal sensor that takes a temperature measurement of an inside of the housing;
a fan that cools the inside of the housing including the motor; and
a controller that controls the fan based on the temperature measurement from the thermal sensor.

2. The bioelectric measurement device according to claim 1, wherein the controller turns the fan on and off based on the temperature measurement.

3. The bioelectric measurement device according to claim 2, wherein the controller turns the fan on if the temperature is higher than a predefined temperature range and turns the fan off if the temperature measurement is within or lower than the predefined temperature range.

4. The bioelectric measurement device according to claim 1, wherein the controller variably controls the fan speed based on based on where the temperature measurement falls relative to the predefined temperature range.

5. The bioelectric measurement device according to claim 1, wherein the controller controls the motor such that the motor output force applied to the conductive tip is automated;
wherein the motor output force is variable, and the controller adjusts the motor output force based on the temperature measurement.

6. The bioelectric measurement device according to claim 1, wherein, when the fan as controlled does not keep the temperature measurement within a predefined temperature range, the controller shuts down the bioelectric measurement device.

7. The bioelectric measurement device according to claim 5, wherein, when the fan, as controlled, does not keep the temperature measurement within a predefined temperature range, and the motor, as controlled, does not maintain a desired motor output force, the controller shuts down the bioelectric measurement device.

8. The bioelectric measurement device according to claim 7, wherein the controller alerts the user about the bioelectric measurement device shutting down.

9. The bioelectric measurement device according to claim 1, wherein the thermal sensor comprises a plurality of thermal sensors disposed at a plurality of locations in the housing to obtain one or more temperature values from each of the plurality of thermal sensors.

10. The bioelectric measurement device according to claim 9, wherein the temperature measurement is an average of the temperature values obtained from the plurality of thermal sensors.

11. The bioelectric measurement device according to claim 1, wherein the housing is a non-conductive housing comprising:
a non-conductive body; and
an isolating hood surrounding the conductive tip to isolate a technician from other elements of the bioelectric measurement device.

12. A method for taking bioelectric measurements with a bioelectric measurement device comprising a housing, a conductive tip disposed at a distal end of the housing, a motor within the housing that applies a motor output force to the conductive tip, a thermal sensor that takes a temperature measurement of an inside of the housing, a fan that cools the inside of the housing including the motor, and a controller that controls the fan based on the temperature measurement from the thermal sensor, the method comprising:
receiving, by the controller, a temperature measurement from the thermal sensor;
comparing the temperature measurement to a predefined temperature range; and
controlling the fan based on the temperature measurement.

13. The method according to claim 12, wherein controlling the fan based on the temperature measurement comprises turning the fan on or off based on the temperature measurement relative to the predefined temperature range.

14. The method according to claim 13, wherein controlling the fan based on the temperature measurement comprises turning the fan on if the temperature is higher than a predefined temperature range and turns the fan off if the temperature measurement is within or lower than the predefined temperature range.

15. The method according to claim 12, wherein controlling the fan based on the temperature measurement comprises variably controlling the fan speed based on where the temperature measurement falls relative to the predefined temperature range.

16. The method according to claim 12, wherein the controller controls the motor such that the motor output force applied to the conductive tip is automated and variable, the method further comprising:
adjusting the motor output force based on the temperature measurement.

17. The method according to claim 12, further comprising:
shutting down the bioelectric measurement device when the fan, as controlled, does not keep the temperature measurement within a predefined temperature range.

18. The method according to claim 12, further comprising:
shutting down the bioelectric measurement device when the fan, as controlled, does not keep the temperature measurement within a predefined temperature range, and the motor, as controlled, does not maintain a desired motor output force.

19. The method according to claim 18, further comprising:
alerting the user about the bioelectric measurement device shutting down.

20. The method according to claim 12, wherein the thermal sensor comprises a plurality of thermal sensors disposed at a plurality of locations in the housing to obtain one or more temperature values from each of the plurality of thermal sensors, and the method further comprises:
calculating an average of the temperature values obtained from the plurality of thermal sensors and using the average as the temperature measurement.
